(19) United States
(12) Patent Application Publication
(10) Pub. No.: US 2006/0121477 A1
(43) Pub. Date: Jun. 8, 2006

(54) Method for detection of micro-metastasis

(76) Inventors: Abraham Hochberg, Jerusalem (IL); Suhail Ayesh, Jerusalem (IL)

Correspondence Address: BROWDY AND NEIMARK, P.L.L.C.;624 NINTH STREET, NW, SUITE 300, WASHINGTON, DC, 20001-5303, US

(21) Appl. No.: 10/527,824

(22) PCT Filed: Sep. 12, 2003
(86) PCT No.: PCT/US03/28807

§ 371 Date: Nov. 9, 2005

Related U.S. Application Data
(60) Provisional application No. 60/409,975, filed on Sep. 12, 2002.

(51) Int. Cl. C12Q 1/68

(52) U.S. Cl. 435/6

(57) ABSTRACT

The present invention concerns a method for the identification of micro-metastasis or residual cancer cells, by identifying the presence of H19 in a sample.

### FIELD OF THE INVENTION

The invention is in the field of cancer detection. More specifically, the invention relates to the detection of micro-metastasis.

### BACKGROUND OF THE INVENTION

The H19 gene is one of the few genes known to be imprinted in humans (Hurst et al., 1996, Nature Genetics 12:234-237). At the very beginning of embryogenesis, H19 is expressed from both chromosomal alleles (DeGroot et al., 1994, Trophoblast 8:285-302). Shortly afterwards, silencing of the paternal allele occurs, and only the maternally inherited allele is transcribed.

H19 is abundantly expressed during embryogenesis, and was first identified as a gene that was coordinately regulated with alpha-fetoprotein in liver by the trans-acting locus raf (Pachnis et al., 1984, Proc. Natl. Acad. Sci. USA 81:5523-5527). Additionally, H19 has been independently cloned by a number of groups using screens aimed at isolating genes expressed during tissue differentiation. For example, Davis et al. (1987, Cell 51:987-1000) identified the mouse homolog of H19 in a screen for genes active early during differentiation of C3H10T1/2cells. Pourier et al. (1991, Development 113:1105-1114) found that murine H19 was expressed during stem cell differentiation and at the time of implantation. Transcription of the human H19 gene was also discovered in differentiating cytotrophoblasts from human placenta (Rachmilewitz et al., 1992, Molec. Reprod. Dev. 32:196-202).

While transcription of H19 RNA occurs in a number of different embryonic tissues throughout fetal life and placental development, H19 expression is down-regulated postnatally. Relatively low levels of H19 transcription have been reported, however, in murine adult muscle and liver (Brunkow and Tilghman, 1991, Genes & Dev. 5:1092-1101). H19 also is activated postnatally in cancer cells. Ariel et al. (1997, Molec. Pathol. 50:34-44) demonstrated H19 expression in a number of tumors arising from the tissues in which it is expressed prenatally. Additionally, these authors found H19 RNA in tumors derived from neural tissues, in particular astrocytoma and ganglioneuroblastoma, that are not known to be associated with H19 expression. Given the large array of cancers expressing H19 RNA, the authors speculated that H19 is an oncofetal RNA and proposed investigating H19 as a tumor marker for human neoplasia.

Both human and murine H19 genes have been cloned and sequenced (Brannan et al., 1990, Molec. Cell. Biol. 10:28-36). Comparison of the human and mouse H19 genes revealed an overall 77% nucleotide sequence identity. Despite this conservation of nucleotide homology between species, very low deduced amino acid sequence identity could be predicted from the open reading frames of the two genes (Id.). Further, although H19 RNA is transcribed by RNA polymerase II, spliced and polyadenylated, it does not appear to be translated. Instead, H19 RNA has been found associated with the 28S cytoplasmic RNA, leading to speculation that H19 RNA may function as an RNA component of a ribonucleoprotein (Id.).

Another function proposed for the H19 gene product is that of a tumor suppressor RNA. Hao et al. (1993, Nature 365:764-767) reported that transfection of two embryonic tumor cell lines, RD and G401, with an H19 expression construct resulted in cell growth retardation, morphological changes and reduced tumorigenicity in nude mice. Such a tumor suppressor activity has been noted as consistent with the observed lethality of ectopic expression in mice (Hao et al., supra) as well as the increased size of mice that are knocked out for the maternal H19 allele (Leighton et al., supra). The proposal that H19 is a tumor suppressor has been controversial, however. Some of the results were reportedly not reproduced, and there may exist another candidate tumor suppressor gene closely linked to H19 (Ariel et al., supra). H19's proposed role as a tumor suppressor also conflicts with the experimental data that H19 is activated in a broad array of tumor cells (see for example Lustig-Yariv et al., 1997, Oncogene 23:169-177).

U.S. Pat. No. 5,955,273 discloses a method for detecting bladder carcinoma in cells or tissue by using a probe that hybridizes to the H19 gene and determining the hybridization of the probe in the bladder itself. This patent is restricted to the identification of bladder cancer at the primary tumor site by hybridization of a probe.

Metastasis spread of cancer begins with the dissociation of cancer cells from the primary tumor. The dissociated cancer cells either settle in, or trespass through, the tissues/organs that they encounter, thus leaving residual or micro-metastasis in the tissues or organs. Detection of the residual cells and micro-metastasis in tissues or organs other than the originating tissues and detection of circulating cancer cells constitutes an important aspect in staging, predicting prognosis, and designing suitable therapy for the cancer patient. However, the tiny size of micro-metastasis and low number of tumor cells, particularly in the circulation and bone marrow, have presented a challenge for their detection in a reliable and sensitive manner. Various techniques have been tried for the detection including fat clearing techniques, serial sectioning and immunohistochemistry Recent studies have shown that molecular detection of micro-metastasis disease from lymph nodes, bone marrow, and the blood circulation can provide very valuable information for the presence of micro-residual disease and its impact on tumor progression and clinical outcomes (CANCER, Principles & Practice of Oncology, 2001, 6^{th} edition, Lippincott Williams & Wilkins, De Vita et al.) The most common types of human cancers bear a considerable risk of systemic recurrence even when they diagnosed and despite curative resection of the primary tumor. Those patients with resected localized cancer who finally progress to lethal metastatic disease would be eligible for adjuvant therapy if reliable prognostic parameters were available to predict individual clinical outcomes. Therefore, systemically disseminated tumor cells have become the subject of intensive research as the presumed seminal precursors of later distant metastasis, which may persist in a state of dormancy for many years. Immunocytochemical techniques based on monoclonal antibodies against cytokeratins and other differentiation markers have been applied to identify rare, disseminated cells of epithelial tumors in bone marrow aspirates of carcinoma patients. The presence of cytokeratin-positive cells correlates with a significantly higher risk of future distant metastasis, as shown for patients with breast cancer and other carcinomas. Nevertheless, the microscopic, preferably double-blinded examination of cytocentrifuged bone marrow cells is laborious and observer dependent, thus complicating routine use.

As an alternative, mRNA transcribed from genes encoding differentiation markers or tumor-associated antigens could be detected in blood, bone marrow, or lymph nodes by sensitive RT-PCR to identify disseminated tumor cells in various types of cancer. However, low-level gene expression in nonmalignant cells appears to limit the specificity of most candidate PCR markers, with only a few exceptions including PSA in prostate cancer.

Several molecular markers for the detection of occult cancer tumor cells (either from micrometastasis or from dissimilated cells) in peripheral blood have been described in literature. Commonly assessed mRNA markers include CK18, CK19, CK20, Mucin-1 (MUC-1), and carcinoembryonic antigen (for breast and colon); EWS-FL11EWS (for Ewing sarcoma, pNET's); ERG, PAX3-FKHR, FAX7-FKHR (for alveolar rhabdomyosarcoma); prostate specific antigen (PSA), prostate membrane specify antigen (prostate cancer); tyrosine hydroxylase, PGP 9.5 (for neuroblastoma), tyrosinase, PG6 9.5. MAGE (for melanoma), alpha-fetoprotein, albumin (for hepatoma); cytokeratins (epithelial) (Methods in Molecular Medicine, Vol 16:Clinical Applications of PCR, Edited by Y. M. D. Lo, Human Press Inc, Susan A, Burchil).

However, recent studies have shown several of these markers to be expressed in normal cells of peripheral blood, lymph nodes, and/or bone marrow yielding false-positive results. More so, many of these molecular markers are also expressed in normal epithelial cells. These findings may contribute to the lack of consistent correlations between any single tumor marker and well-known clinical and pathological prognostic factors. Currently there is no consensus recommendation for the routine use of molecular markers in monitoring disease detection in blood or other body fluids.

Thus there is a need to identify a tumor marker that is specific and sensitive enough to enable detection of minute amounts of cancer cells, as can be found in micro-metastasis or in residual cancer cells. Preferably the marker should not be expressed in non-malignant cells or expressed only in very low level in non-malignant cells.

### SUMMARY OF THE INVENTION

The present invention is based on the surprising finding that by detecting the presence of H19 mRNA in a cell-containing sample, obtained from a cancer patient, it is possible to detect the presence of minute amounts of circulating cancer cells, either in body fluids or in tissues other than from the originating tissue. The detection of H19 RNA thus enables the detection of the presence of micrometastasis, or residual cancer cells, in a very sensitive manner.

The present invention thus enables the identification of cells from solid tumors that became dissociated from the originating tissue or organ (hereinafter “the primary tumor site”), spontaneously or due to a medicinal manipulation such as surgical removal of the originating tumor (such spontaneously/mechanically dissociated cells being referred to as “residual cells”), or cells that became dissociated from the primary tumor site due to active re-colonization processes (referred to as “micro metastasis”) by identifying the presence of H19 RNA in a sample containing those cells.

Thus the present invention concerns a method for the detection, in a patient suspected of having cancer, of the presence of residual cancer cells or micro-metastasis originating from solid tumors, the method comprising;
(a) obtaining from the patient a cell-containing specimen of a sample selected from:
   (1) body fluids,
   (2) a rinse fluid that was in contact with the primary tumor site,
   (3) tissues, or organs other than the tissue primary tumor site,
(b) detecting the presence of H19 RNA in the above specimen, a presence beyond that of a standard threshold, indicating the presence of residual cancer cells, and/or micro-metastasis originating from solid tumors in the patient.

In accordance with a preferred embodiment the method is carried out by the simultaneous detection of the H19 RNA and at least one additional tumor marker as will be explained below. By one embodiment the tumor marker may be an mRNA tumor marker. Preferably the tumor marker is a tissue specific tumor marker.

By another aspect the present invention concerns methods for evaluation of the level, or amount of the residual cancer cells or cancer cells from micrometastasis originating from solid tumors, in a patient so as to receive some sort of indication of the tumor load. At times it is not sufficient merely to know, in a binary yes/no fashion, whether there are residual cancer cells or micrometastasis un a sample obtained from a patient. Sometimes the quantified determination of the amount/level of these cells in the patient (sample) is crucial for establishing the prognosis of the patient and determining the optimal course of treatment.

Thus the present invention concerns a method for the determination, in a patient suspected of having cancer, of the amount of residual cancer cells or cancer cells from micro-metastasis originating from solid tumors, the method comprising:
(a) obtaining from the patient a cell-containing specimen of a sample selected from:
   (1) body fluids,
   (2) a rinse fluid that was in contact with the primary tumor site,
   (3) tissues, or organs other than the tissue primary tumor site,
(b) quantifying the amount of H19 RNA in the above specimen, and determining the amount of cancer cells by comparing the amounts of the quantified H19 mRNA in the sample to standard calibration curve of H19 mRNA as a function of the number of cancer cells, thereby determining the amount residual cancer cells or cancer cells from micrometastasis in the patient.

The term: “residual cancer cells” refers to cells that became dissociated from the primary tumor site in general for example during spontaneous processes of shedding, and in particular to cells that became detached from the primary tumor site after surgical removal of the primary tumor, typically due to mechanical disintegration of the tumor or due to failure to fully remove all the malignant tissue. The term also concerns cancer cells that do not feature physiological characteristics of cells undergoing metastasis (such as the ability to breakdown extracellular tissue and penetrate through tissue), but are rather present either in the vicinity of the primary tumor site or in body fluids due to physical detachment from the primary tumor.

The term “micro metastasis” refers to cells that became dissociated from the primary tumor and either settle, trespass or circulate through the tissues they encounter. Typically, these cells are metastatic cells that feature active metastatic processes such as penetration through extracellular matrix, etc.

The term “solid tumors” refers to any tumor which is not from hematopoietic origin.

In particular this term refers to: carcinoma, sarcoma, adenoma, hepatocellular carcinoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, lymphangiosarcoma, synovioma, Ewing's tumor, leiomyosarcoma, rhabdotheliosarcoma, colon carcinoma, pancreatic cancer, breast cancer, ovarian cancer, prostate cancer, squamous cell carcinoma, basal cell carcinoma, adenocarcinoma, renal cell carcinoma, hematoma, bile duct carcinoma, melanoma, choriocarcinoma, seminoma, embryonal carcinoma, Wilms' tumor, cervical cancer, testicular tumor, lung carcinoma, small lung carcinoma, bladder carcinoma, epithelial carcinoma, glioma astrocytoma), medulloblastoma, craniopharyngioma, ependymoma, pinealoma, retinoblastoma, multiple myeloma, rectal carcinoma, cancer of the thyroid, head and neck cancer, cancer of the endometrium.

Preferably the cancer is selected from: breast cancer, colon cancer, lung cancer, bladder cancer, melanoma and liver cancer.

The term “body fluid” refers to urine, blood, cerebro-spinal fluid, lymph fluid, lung embolism, sperm, saliva, synovial fluids, and feces (which are diluted in fluids and thus considered as a body fluid).

The term “a rinse fluid that was in contact with the primary tumor site” refers to externally introduced fluid, such as saline, used to flush away epithelial cells from a body cavity such as the uterus, vagina, bladder, intraperitoneal cavity, gastrointestinal tract, lungs etc., so that the re-collected rinse fluid contains epithelial cells lining the body cavity.

The term “organ or tissue other than the primary tumor site” refers to a tissue or organ in which the cancer cells re-colonized after dissociating from the primary tumor site, and in particular this term refers to lymph nodes a, bone marrow, peripheral stem cell harvests, lung and liver samples (obtained by needle biopsies) where cells from the primary tumor re-colonize in the metastatic process.

The term “primary tumor site” refers to the site, organ or tissue were the cancer cells of the solid tumor first originated.

The term “H19 RNA” refers to Accession Number AF087017. Homo sapiens H19, BC006831.

The body sample of fluid, tissue, organ or rinse fluid is obtained by any routine procedure such as drawing blood, collecting bone marrow, obtaining liver biopsies, rinsing the body cavity (for example bladder) with saline and re-collecting the rinse fluid, etc. The cells are separated therefrom according to the type of sample, typically if the sample is liquid, by centrifugation, if the sample is a lymph mode the cells may be merely disintegrated for example by ultrasonic procedures.

Then the presence of H19 RNA in the cells is then detected. The detection may be by any methods used in the art for the detection of RNA in a cell-containing sample such as in situ hybridization with a detectable label, for example, with a complementary sequence containing a detectable moiety (fluorescent, radioactive, chromatophoric moiety, etc). In such a case of in situ hybridization there is no need to extract the DNA from the cells. However various amplification methods, which, are sensitive enough to detect to minute amounts of RNA are preferable. Such methods include, PCR, RT-PCR, in situ PCR, in situ RT-PCR 9all the above referring also to “nested” PCR, and nested RT-PCR), LCR (ligase chain reaction) and 3SR (self sustained sequence replication). In accordance with a preferred embodiment RT-PCR and nested RT-PCR are used. The amplification products are identified by methods used in the art such as by separation on a gel.

Typically the presence or absence of the amplified RNA molecule is determined by the presence or absence of an amplification curve. Those samples showing no amplification curve are scored as negative. Samples showing an amplification curve will be scored as positive and quantified by determining the cycle threshold and comparing it to a standard curve run with each assay. Positive and negative controls are also run with each assay.

The presence of the H19 RNA is determined by comparison of the detected level of the H19 in the sample to a standard threshold level. As some amplification techniques are capable of detecting even the presence of a single RNA molecule, which may be present as a residual molecule or as contamination of the sample, obviously the amplification results have to be calibrated. By a most extreme example, a negative result is considered when no amplification curve is present, i.e., there is virtually no H19 mRNA in the sample.

Calibration may take place by various manners. Typically a calibration curve for the amplification procedure is prepared using known amounts of H19 RNA that are added to the sample. For example, known amounts of H19 RNA added to the blood, saline, etc., and then detected using any one of the above techniques, preferably RT-PCR, resulting in a calibration curve wherein a known amount of RNA can be associated to an RT-PCR results. This can be done once for establishing an “external” standard curve, i.e., creating once a curve with known amounts of H19 and using this curve in all subsequent assays. Alternatively the “standard” curve can be established again in each run. The curve can also be used to qualify the H19 to establish the correlation between the amounts of H19 and the amount/level of cancer cells.

Then the amounts and the calibration curve are clinically correlated to actual t samples obtained from diagnosed patients to establish a threshold level of RNA (or rather a RT-PCR amplification result for the specific assay conditions) for each type of sample (for example, taking into consideration the amount of residual or micro-metastatic cells in a body fluid vs. a lymph node, for example) and for each type of cancer.

Therefore the present invention concerns a method, wherein the standard threshold RNA level is established by:
(a) performing an RNA detection assay on externally added known and varying amounts of H19 RNA in a medium selected from: a body fluid, a rinse fluid, bone marrow lymph node, lung or liver tissue to produce a calibration curve showing the level of reading of the RNA detection assay as a function of the amounts of known H19 RNA in the medium;
(b) correlating the amounts of H19 in the calibration curve of (a) above, to the H19 RNA levels obtained from a plurality of diagnosed patients of a specific tumor and the H19 RNA levels of plurality healthy controls, when using the same type of RNA detection assay, and the same type of sample as used in (a) above
(c) defining an H19 level that differentiates between the amounts of H19 in the diagnosed patients and the healthy controls;
   said differentiating H19 level being the standard threshold H19 level.

Above this standard threshold level, H19 level is considered as “present” so that the sample is declared as containing residual cancer cells or micro-metastasis. In practice many times the threshold standard level is nil, i.e., complete lack of H19 molecules.

The present invention also provides a method for creating a calibration curve for determining the amount of residual cancer cell or cancer cells from micrometastasis the method comprising:
(a) obtaining known and varying amounts of cancer cells from a specific cancer;
(b) determining the level of H19 mRNA for each known amount of cells,
   thereby establishing a curve wherein the level of H19 is a function of the number of the specific cancer cells.

Preferably, in accordance with the invention the H19 is detected together with at least one other tumor marker. The tumor marker can be detected in any sort of cell containing sample as described above. The marker may be a protein, a peptide, an mRNA or DNA molecule. Preferably the marker is an mRNA marker and most preferably an mRNA tissue specific tumor marker, most preferably with a plurality of RNA tumor markers so as to increase the reliability of the detection method of the invention.

Examples of RNA tumor markers are: CEA, CK19, CK20, c-Met, MAGE-A3, β-hCG, GalNAc-T, CK18, Mucin-1 (MUC-1), and carcinoembryonic antigen (for breast and colon); EWS-FL11EWS (for Ewing sarcoma, pNET's); ERG, PAX3-FKHR, FAX7-FKHR (for alveolar rhabdomyosarcoma); prostate specific antigen (PSA), prostate membrane specific antigen (prostate cancer); tyrosine hydroxylase, PGP 9.5 (for neuroblastoma), tyrosinase, PG6 9.5. MAGE (for melanoma), alpha-fetoprotein, albumin (for hepatoma); cytokeratins (epithelial cells)

The method of the invention can be used to detect micro-metastasis or residuals where all other imaging techniques are not sensitive enough to detect and by this help and establish the prognosis of the patient and decide of the best course of treatment.

Where the primary tumor site/organ is removed the method may be used to assess the amount of dissociated cells, or circulating metastatic cell before the removal, immediately following the surgery, and after a time laps from the surgery so as to determine the success of the tumor removal and to help decide whether another surgical procedure, or another anticancer therapy are required.

The present invention also concerns a kit for use in the above method. Typically the kit contains reagents for mRNA detection and more specifically reagents for RT-PCR including primers and amplification reagents. The kit further comprised means foe determining the amounts of amplified mRNA and some sort of standard calibration curve, either set once as an “external” standard or alternatively re-created again with suitable control in each assay.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows in situ hybridization of an H19 labeled probe in cells from the urine of a bladder carcinoma patients

FIG. 2 shows a gel of the RT-PCR amplification products of H19 mRNA obtained from blood of 4 colon cancer patients (Samples B1-B4). M is a marker. Samples B1 and B2 have positive H19 expression.

FIG. 3 shows a gel of the RT-PCR amplification products of H19 mRNA obtained from lymph nodes of 7 breast cancer patients (Samples L1-L7). Samples L5 and L6 have positive H19 expression.

### DETAILED DESCRIPTION OF THE INVENTION

I. Experimental Procedures

(a) RNA Extraction (STAT-60) from Blood

1. hemolysis of RBCs
2. lysis by using Stat-60 1 ml/5-10*106 cells
3. store for 5 min at RT
4. add 0.2 ml (200 μl) chloroform (0.2 ml/l ml Stat-60)
5. shake vigorously for 15 sec
6. store for 2-3 min at RT. (then put in the freezer for 2 min)
7. centrifuge at 12,000 g for 15 min at 4° C.
8. transfer the upper (60%) aqueous phase to a fresh tube
9. add 0.5 ml isopropanol (per 1 ml Stat), mix 30-40 times
10. store at RT for 5-10 min (then put in the freezer for 5 min)
11. centrifuge at 12,000 g for 10 min at 4° C.
12. remove the supernatant and wash RNA pellet
13. add 1 ml of 75% ethanol (per 1 ml Stat-60)
14. centrifuge at 7,000 g for 5 min at 4° C.
15. air dry the pellet
16. dissolve the pellet in 50 μl 2.5% HPRI-DEPC treated D.W (7.5 HPR1 in 300 DEPC treated D.W), vortex or pipette
   (b) First-Strand cDNA Synthesis Using M-MLV RT for RT-PCR:

A 20-μl reaction volume is used for 1 ng-5 μg of total RNA or 1 ng-500 ng of mRNA. The following components are added to a nuclease-free microcentrifuge tube:
1 μl Oligo(dT)12-18 (500 μg/ml)
1 ng to 5 μg total RNA
1 μl 10 mM dNTP Mix (10 mM each DATP, dGTP, dCTP and dTTP at neutral pH)
sterile, distilled water to 12 ul

Heat mixture to 65° C. for 5 min and quick chill on ice. Collect the contents of the tube by brief centrifugation and add:
4 μl 5× First-Strand Buffer
2 μl 0.1 M DTT
1 μl RNASEOUT Recombinant Ribonuclease Inhibitor (40 units/μl) (when using less than 50 ng of starting RNA, the addition of RNASEOUT is essential.)

Mix contents of the tube gently and incubate at 37° C. for 2 min. Add 1 μl (260 units) of M-MLV RT, mix by pipetting gently up and down. Incubate 50 min at 37° C. Inactivate the reaction by heating at 70° C. for 15 min. The cDNA can now be used as a template for amplification in PCR.

| | Primer's | | | | | No. | Gel | Tube |
|---|---|---|---|---|---|---|---|---|
| Primers | Volume | cDNA | Formamide | Program | Tm | Cycles | Conc. | Volume |
| H19 F, | 1 ul each | | 1.5% | H19R1 | 58° C. | 38 | 2% | 25 μl |
| H19 R | (0.1 μg/μl) | | | | | | | |

PCR Program (H19R1):

| 98° C. | 2 min |
|---|---|
| 98° C. | 15 sec |
| 58° C. | 30 sec |
| 72° C. | 40 sec |
| Go to step 2- 38× | |
| 72° C. | 5 min |
| 4° C. | 30 min |
| 12° C. | 24 hr |

The primers sequence

| | |
|---|---|
| H19 Forward: CCGGCCTTCCTGAACA | (SEQ ID NO:1) |
| H19 Reverse: TTCCGATGGTGTCTTTGATGT | (SEQ ID NO:2) |

(c) RT-PCR Quantification Techniques

H19 RT-PCR may be quantified using the teaching of Milligan et al, EMBO reports, Vol 3, 774-779, 2002, which indicates also the manner for real time RT-CR.

Other references concerning quantification of RT-PCR in real time include:
1) Roche Molecular Biochemical rapid real time quantification of RT-PCR adapted from a poster presented in annual meeting of the Association of Molecular Pathology, St. Louis, Mo., USA, Nov. 4-7 1999
2) Idaho Technology's Quantification on the LightCycler® Instrument www.idahotec.com/lightcycler_u/lectures/quantification_on_lc.htm
3) Quantification of bcr/able transcripts by RT-PCR www.aruplab.com/testbltn/bcr-abl.htm
   (d) In Situ Hybridization with an H19 Probe

In situ hybridization with a labeled H19 probe was performed as described in Ariel et al., J. Clin. Pathol: Mol Pathol. 1998: 51, 21-25.

(e) Sentinel Node Biopsy

Biopsies from the sentinel nodes of breast cancer patients were obtained as described in Tafra et al. Annals of Surgery, 233 (1), 51-59, (2001). Half the lymph node was sent to normal pathology for evaluation and have was mechanically disintegrated using a tissue homogenizer and the H10 level determined.

### EXAMPLE 1

### Detection of H19 in Bladder Rinse Fluid

Voided urine was taken from patient with bladder carcinoma (DIG-H19). The exfoliated cells in the urine were separated from the liquid and underwent in situ hybridization with a radioactive H19 probe as described in I(c) in experimental procedures above.

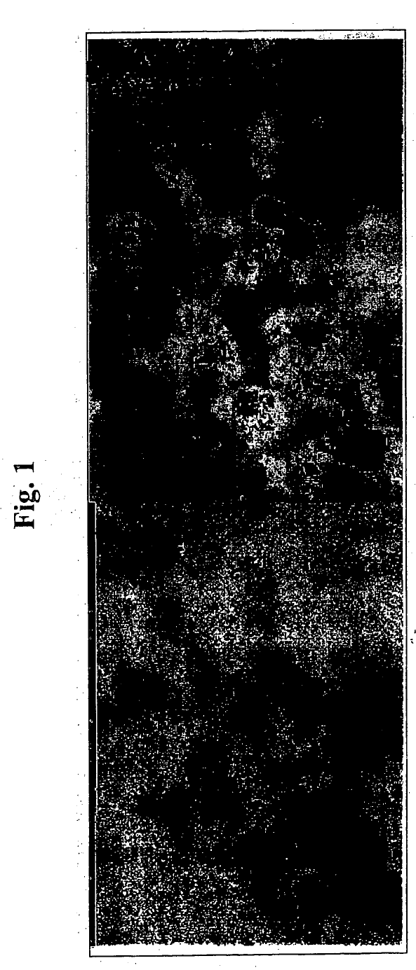

The results of the in situ hybridization are shown in FIG. 1. As can be seen the cells present in the urine of cancer patient reacted significantly with the labeled probe, while normal urine (data not shown) did not hybridize with the probe.

### EXAMPLE 2

### Detection of H19 in Blood Samples of Colon Patients

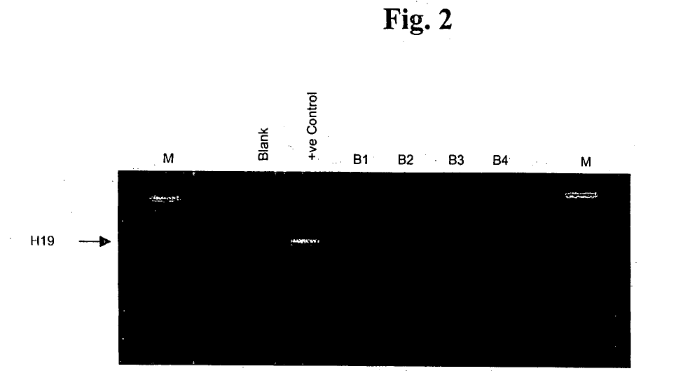

Blood from 4 diagnosed colon cancer patients was colleted and prepared as in I(a) above and the H19 mRNA was amplified by RT-PCR as disclosed in I(b) above. The amplification products were separated on a gel and the results are shown in FIG. 2. As can be seen patients B1 and B2 were strongly positive for H19 expression (as compared to blank) while patient B3 showed a week expression of H19, indicating that 3 out of the 4 colon cancer patients had H19 expression in a detectable level.

### EXAMPLE 3

### Detection of H19 in Lymph Nodes Obtained from Breast Cancer Patients

Sentinel lymph nodes were obtained from breast cancer patients as described in I(e) above. The RT-PCR was performed on the extracted mRNA as described in I (b) above and the amplification results were separated on a gel.

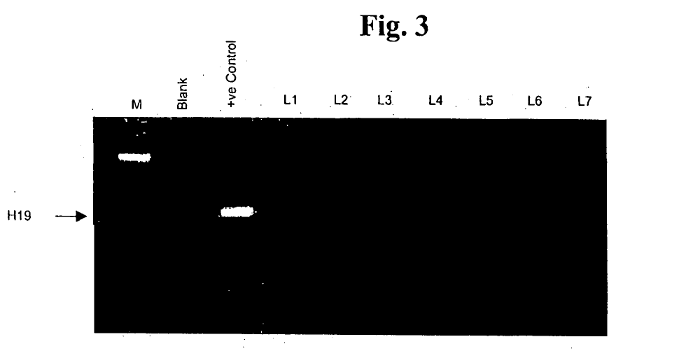

The results are shown in FIG. 3. As can be seen patients L5 and L6 were tested positive for H19 expression indicating that H19 detection can be carried in a lymph node sample.

1. A method for the detection in, a patient suspected of having cancer, of the presence of residual cancer cells or micro-metastasis originating from solid tumors, the method comprising:
obtaining from the patient a cell-containing specimen of a sample selected from:
1) body fluids;
2) a rinse fluid that was in contact with the primary tumor site,
3) tissues, or organs other than the tissue primary tumor site, and
detecting the presence of H19 RNA in the above specimen, a presence beyond that of a standard threshold indicating the presence of residual cancer cells, and/or micro-metastasis originating from solid tumors in the patient.

2. A method for the determination, in a patient suspected of having cancer, of the amount of residual cancer cells or cancer cells from micro-metastasis originating from solid tumors, the method comprising:
obtaining from the patient a cell-containing specimen of a sample selected from:
1) body fluids;
2) a rinse fluid that was in contact with the primary tumor site, and
3) tissues, or organs other than the tissue primary tumor site; and
quantifying the amount of H19 RNA in the above specimen, and determining the amount of cancer cells by comparing the amounts of the quantified H19 mRNA in the sample to standard calibration curve of H19 mRNA as a function of the number of cancer cells, thereby determining the amount residual cancer cells or cancer cells from micrometastasis in the patient.

3. A method according to claim 1, further comprising the steps of:
detecting the presence of at least one additional tumor marker in a cell-containing specimen of samples (1) to (3) above; a presence of both said additional tumor marker and H19 RNA beyond that of a standard threshold, indicating the presence of residual cancer cells and/or micro-metastasis from solid tumors in the patient.

4. A method according to claim 3 wherein the additional tumor marker is mRNA tumor marker.

5. A method according to claim 4 wherein the tumor marker is selected from: CK18, CK19, CK20, Mucin-1 (MUC-1), carcinoembryonic antigen; EWS-FL11EWS; ERG, PAX3-FKHR, FAX7-FKHR; prostate specific antigen (PSA), prostate membrane specific antigen; tyrosine hydroxylase, PGP 9.5, tyrosinase, PG6 9.5. MAGE (for melanoma), alpha-fetoprotein, albumin; cytokeratins.

6. A method according to claim 1, wherein the solid tumor is selected from: carcinoma, sarcoma, adenoma, hepatocellular carcinoma, hepatoblastoma, rhabdomyosarcoma, esophageal carcinoma, thyroid carcinoma, ganglioblastoma, fibrosarcoma, myxosarcoma, liposarcoma, cohndrosarcoma, osteogenic sarcoma, chordoma, angiosarcoma, endotheliosarcoma, lymphagiosarcoma, synovioama, Ewing's tumor, leimyosarcoma, rhabdotheliosarcoma, colon carcinoma, pancreatic cancer, breast cancer, ovarian cancer, prostate cancer, squamous cell carcinoma, basal cell carcinoma, adenocarcinoma, renal cell carcinoma, hematoma, bile duct carcinoma, melanoma, choriocarcinoma, seminoma, embryonal carcinoma, Wilms' tumor, cervical cancer, testicular tumor, lung carcinoma, small lung carcinoma, bladder carcinoma, epithelial carcinoma, glioma astrocyoma), medulloblastoma, craniopharyngioma, ependynoma, pinealoma, retinoblastoma, multiple myeloma, rectal carcinoma, cancer of the thyroid, head and neck cancer, cancer of the edometrium.

7. A method according to claim 1, wherein the body fluid is selected from: urine, blood, cerebro-spinal fluid, lymph fluid, lung embolism, sperm synovial fluid, saliva, and feces.

8. A method according to claim 1, wherein the rinse fluid was obtained by rising a body cavity selected from: uterine, vagina, bladder, intraperitoneal cavity, gastrointestinal cavity and lung.

9. A method according to claim 1, wherein said organ or tissue other than the primary tumor site is lymph node, bone marrow, peripheral stem cell harvests, lung or liver biopsies.

10. A method according to claim 1, wherein the RNA is detected in the sample by a method selected from: PCR, RT-PCR, in situ PCR, in situ RT-PCR, LCR and, 3SR, and hybridization with a probe comprising a detectable moiety.

11. A method according to claim 1, wherein the standard threshold RNA level is established by:
(a) performing an RNA detection assay by adding varying and known amounts of H19 RNA to a sample selected from: a body fluid, a rinse fluid, bone marrow, lymph node, liver or lung tissue to produce a calibration curve showing the level of reading of the RNA detection assay as a function of the amounts of known H19 RNA;
(b) correlating the amounts of H19 in the calibration curve of (a) above, to the H19 RNA levels obtained from a plurality of diagnosed patients of a specific tumor, and the H19 RNA levels of plurality healthy controls, when using the same sample and same RNA detection assay as used in (a) above; and
(c) defining an H19 level that differentiates between the amounts of H19 in the diagnosed patients and the healthy controls, said differentiating H19 level being the standard threshold H19 level.

12. (canceled)

13. A method according to claim 2, wherein the solid tumor is selected from: carcinoma, sarcoma, adenoma, hepatocellular carcinoma, hepatoblastoma, rhabdomyosarcoma, esophageal carcinoma, thyroid carcinoma, ganglioblastoma, fibrosarcoma, myxosarcoma, liposarcoma, cohndrosarcoma, osteogenic sarcoma, chordoma, angiosarcoma, endotheliosarcoma, lymphagiosarcoma, synovioama, Ewing's tumor, leimyosarcoma, rhabdotheliosarcoma, colon carcinoma, pancreatic cancer, breast cancer, ovarian cancer, prostate cancer, squamous cell carcinoma, basal cell carcinoma, adenocarcinoma, renal cell carcinoma, hematoma, bile duct carcinoma, melanoma, choriocarcinoma, seminoma, embryonal carcinoma, Wilms' tumor, cervical cancer, testicular tumor, lung carcinoma, small lung carcinoma, bladder carcinoma, epithelial carcinoma, glioma astrocyoma), medulloblastoma, craniopharyngioma, ependynoma, pinealoma, retinoblastoma, multiple myeloma, rectal carcinoma, cancer of the thyroid, head and neck cancer, cancer of the edometrium.

14. A method according to claim 2, wherein the body fluid is selected from: urine, blood, cerebro-spinal fluid, lymph fluid, lung embolism, sperm synovial fluid, saliva, and feces.

15. A method according to claim 2, wherein the rinse fluid was obtained by rising a body cavity selected from: uterine, vagina, bladder, intraperitoneal cavity, gastrointestinal cavity and lung.

16. A method according to claim 2, wherein said organ or tissue other than the primary tumor site is lymph node, bone marrow, peripheral stem cell harvests, lung or liver biopsies.

17. A method according to claim 2, wherein the RNA is detected in the sample by a method selected from: PCR, RT-PCR, in situ PCR, in situ RT-PCR, LCR and, 3SR, and hybridization with a probe comprising a detectable moiety.
